(19) United States
(12) Patent Application Publication
(10) Pub. No.: US 2026/0102450 A1
(43) Pub. Date: Apr. 16, 2026

(54) COMPOSITION AND FOOD SUPPLEMENTARY FOR SARCOPENIA AND PERIODONTAL DISEASES CONTAINING ALDER AND ELM COMPLEX EXTRACTS AS ACTIVE INGREDIENTS

(71) Applicant: DR. OREGONIN INC., Gangwon-do (KR)

(72) Inventor: Sun Eun Choi, Gangwon-do (KR)

(21) Appl. No.: 19/182,397

(22) Filed: Apr. 17, 2025

(30) Foreign Application Priority Data
Oct. 10, 2024 (KR) 10-2024-0137792

(51) Int. Cl. A61K 36/185; A61K 31/7028; A61K 31/7048; A61P 1/02; A61P 21/00

(52) U.S. Cl. CPC A61K 36/185; A61K 31/7028; A61K 31/7048; A61P 1/02; A61P 21/00

(57) ABSTRACT

The present disclosure provides a pharmaceutical composition for preventing and treating sarcopenia and periodontal disease, comprising extracts of Alnus japonica and Elm japonica as active ingredients, wherein the Elm japonica extract comprises catechin 7-O-β-D-apiofuranoside (catechin 7-O-β-D-apiofuranoside), wherein a weight ratio of the extract of Alnus japonica and the extract of Elm japonica is 3:7 to 7:3.

### CROSS-REFERENCE TO RELATED APPLICATION

This application claims priority of Korean Patent Application No. 10-2024-0137792, filed on Oct. 10, 2024, in the KIPO (Korean Intellectual Property Office), the disclosure of which is incorporated herein entirely by reference.

### BACKGROUND OF THE INVENTION

### Field of the Invention

The present invention relates to a therapeutic agent and health functional food for preventing and treating sarcopenia and periodontal disease comprising extracts of Alnus japonica and Elm japonica as active ingredients, and more particularly, to a pharmaceutical composition and health functional food comprising a complex extract of Alnus japonica and Elm japonica as an active ingredient for treating or preventing myopathy and periodontal disease.

### Description of the Related Art

Muscles may be divided into skeletal muscles, smooth muscles, and cardiac muscles in terms of structure or function. Among them, skeletal muscles are 600 voluntary muscles that are directly under the skin of the hands, feet, chest, and stomach and attached to the bones through the whole body's bones or tendons. suitable for moving or supporting bone through contraction. The contraction is caused and controlled by a neural signal. It accounts for 40-50% of the body weight and functions to maintain body temperature and generate energy. The micromyofibrils actin and myosin are regularly arranged so that a horizontal pattern can be observed on a microscope.

Skeletal muscle fibers are divided into three categories: Type I, Type IIa, and Type IIb according to the content of mitochondria. A posture support bar, which is made of red geotextile and maintains the posture thereof by maintaining a weak force for a long time, is called Type I. It is a muscle suitable for exercise such as aerobic long-distance running because of its high mitochondrial content. Among the fast root fibers, those having the characteristics of slow root fibers are called Type IIa.

When making movements, muscles made of white fast muscle fibers are used, which is called active muscles and is classified as Type IIb. It is a muscle suitable for exercise such as anaerobic short-distance running because of its low mitochondrial content. These skeletal muscle fibers are distributed in different proportions in each body part.

Muscle atrophy refers to the anti-anabolic and catabolic action of unbalanced muscle fibers. Here, muscle atrophy refers to the size and mass loss of muscle cells and muscle tissue when the muscle is not used due to aging, a disease state (excessive exposure to stress hormones, cancer, sepsis, starvation, etc.), and a decrease in activity such as pathological life. When muscle atrophy occurs, muscle strength for physical activity is weakened, and a vicious cycle of musculoskeletal degeneration begins. A decrease in walking speed and a decrease in grip strength are the main symptoms and indicators of a decrease in muscle mass, and may lead to falls, fractures, joint damage, metabolic disorders, and cardiovascular diseases.

Glucocorticoids in our body are believed to cause molecular biological changes in muscle fibers and is directly or indirectly involved in anti-assimilation and catabolism Dexamethasone, which is a Glucocorticoids-based compound, serves to inhibit PI3K/Akt/mTOR pathway as an anti-anabolic action, and inhibits the activities of 4E-BP1 and S6K1, which are downstream effectors, thereby preventing the operation of eIF4G (Eukaryotic translation initiation factor 4G) and eIF4E (Eukaryotic translation initiation factor 4E). This inhibits the mRNA translation process for protein synthesis, which is shown as inhibiting the synthesis of myofibers and muscle fiber atrophy resulting from protein degradation.

Dexamethasone also induces muscle atrophy by causing synthesis inhibition and proteolysis of muscles. This expresses atrogene genes (Atrogin-1, MuRF-1) that induce muscle atrophy according to a mechanism leading to “PI3K/Akt→FOXO activation and GSK3 inactivation”, and these genes induce proteolysis represented by ubiquitin-proteasome system. Therefore, it is necessary to develop a material for preventing and treating sarcopenia, which is a disease in which skeletal muscle is reduced.

Meanwhile, the periodontal disease refers to any disease that occurs in the periodontal tissue and is divided into gingivitis and periodontitis according to the degree of the disease. Among them, gingivitis is a periodontal disease that is relatively light and recovers quickly, and it refers to a form in which inflammation is limited to the gums, that is, soft tissues, and periodontitis refers to a case in which inflammation has progressed to the periphery of the gums and gums bones. Such periodontal disease is not only a main cause of tooth loss after senile age, but also a high association with systemic diseases such as dementia, arteriosclerosis, myocardial infarction, and stroke is reported, and thus interest in prevention and treatment of periodontal disease is increasing in modern society entering an aging society.

Ulmus is a deciduous or semi-deciduous tree belonging to the Elm family, and can be seen in the northern hemisphere from Siberia to Indonesia, Mexico, and Japan. The Elm japonica includes about 30 to 40 kinds of trees, and seven kinds of Elm japonica trees, Elm japonica, Ulmus davidiana, Ulmus davidiana, Ulmus parvifolia, Ulmus pumila, Ulmus laciniata, and Ulmus macrocarpa inhabit Korea. Elm is medicinal for all of bark, leaves, flowers, fruits and seeds of elm, and ulmi cortex and ulmi cortex, which are typically stem and root bark of Ulmus parvifolia, Elm japonica, and Ulmus macrocarpa, are known for diuretic activity, anti-inflammation, insomnia resolution, constipation prevention, digestive stimulation, and antioxidant effects.

However, there has not been a specific report on the prevention and treatment characteristics of periodontal diseases such as gingivitis of a plant extract of the genus Elm japonica and an active material thereof.

### SUMMARY OF THE INVENTION

Accordingly, the problem to be solved by the present invention is to provide a therapeutic agent and health functional food for preventing and treating sarcopenia and periodontal disease at the same time.

To solve the above problems, the present invention provides a pharmaceutical composition for preventing and treating sarcopenia and periodontal disease, which comprises extracts of Alnus japonica and Elm japonica as active ingredients at the same time.

In an exemplary embodiment of the present invention, the Elm japonica extract includes catechin 7-O-β-D-apiofuranoside.

In one embodiment of the present invention, the Alnus japonica extract comprises oregonin.

In one embodiment of the present invention, the weight ratio of the Alnus japonica and Elm japonica extract is 3:7 to 7:3.

The present invention also provides a health functional food for preventing and alleviating periodontal diseases comprising extracts of Alnus japonica and Elm japonica as active ingredients.

In an exemplary embodiment of the present invention, the Elm japonica extract includes catechin 7-O-β-D-apiofuranoside.

In one embodiment of the present invention, the Alnus japonica extract comprises oregonin.

In one embodiment of the present invention, the weight ratio of the Alnus japonica and Elm japonica extract is 3:7 to 7:3.

The present invention also provides a feed containing extracts of Alnus japonica and Elm japonica as active ingredients at the same time.

In an exemplary embodiment of the present invention, the Elm japonica extract includes catechin 7-O-β-D-apiofuranoside.

In one embodiment of the present invention, the Alnus japonica extract comprises oregonin.

The present invention also relates to a toothpaste or mouthwash comprising the extracts of Alnus japonica and Elm japonica as active ingredients at the same time.

According to the present invention, the extract comprises Alnus japonica and Elm japonica as active ingredients at the same time, thereby having effects of reducing muscle tone and preventing and treating periodontal diseases.

### BRIEF DESCRIPTION OF THE DRAWINGS

The above and other features and advantages will become more apparent to those of ordinary skill in the art by describing in detail exemplary embodiments with reference to the attached drawings, in which:

FIG. 1 is a flowchart illustrating a method for obtaining a complex extract of Ulmus macrocarpa and Alnus japonica according to an embodiment of the present invention.

FIG. 2 is a result of analyzing the content of oregonin of Chemical Formula 1 for each extract.

FIG. 3 is a result of analyzing the content of catechin glycosides of Chemical Formula 2 in each extract.

FIG. 4 is a result of analyzing the effect of inhibiting the expression level of TNF-α of the extract according to the present invention by using lysozyme and batamine C, which are major pharmaceutical components of the existing commercialized therapeutic agent for periodontal diseases (Irgata), as controls.

FIG. 5 is a result of an analysis of inhibition of TNF-α expression with a composite extract according to the present invention compared to a Alnus japonica alone and an Elm japonica alone.

FIG. 6 shows the results of analysis of the IL-6 expression level of the extract according to the present invention using lysozyme and batamine C, which are the main pharmaceutical ingredients of the existing commercialized therapeutic agent for periodontal disease (Irgata), as controls.

FIG. 7 shows the results of analyzing the expression level of IL-6 having a composite extract according to the present invention as compared with a Alnus japonica alone and an Elm japonica alone.

FIG. 8 shows the results of analysis of the expression level of IL-1B of the extract according to the present invention using lysozyme and batamine C, which are the main pharmaceutical ingredients of the existing commercialized therapeutic agent for periodontal disease (Irgata), as controls.

FIG. 9 shows the results of analysis of the expression level of IL-1β having a composite extract according to the present invention as compared with a Alnus japonica alone and an Elm japonica alone.

FIGS. 10 to 14 show results of analyzing cell viability of TM1 to TM5, respectively.

FIGS. 15 to 19 show results of analyzing effects of TM1 to TM5 on apoptosis, respectively.

FIG. 20 shows the results of the diameter of muscle cells for dexamethasone-treated muscular cells.

In the following description, the same or similar elements are labeled with the same or similar reference numbers.

### DETAILED DESCRIPTION

The present invention now will be described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes”, “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. In addition, a term such as a “unit”, a “module”, a “block” or like, when used in the specification, represents a unit that processes at least one function or operation, and the unit or the like may be implemented by hardware or software or a combination of hardware and software.

Reference herein to a layer formed “on” a substrate or other layer refers to a layer formed directly on top of the substrate or other layer or to an intermediate layer or intermediate layers formed on the substrate or other layer. It will also be understood by those skilled in the art that structures or shapes that are “adjacent” to other structures or shapes may have portions that overlap or are disposed below the adjacent features.

In this specification, the relative terms, such as “below”, “above”, “upper”, “lower”, “horizontal”, and “vertical”, may be used to describe the relationship of one component, layer, or region to another component, layer, or region, as shown in the accompanying drawings. It is to be understood that these terms are intended to encompass not only the directions indicated in the figures, but also the other directions of the elements.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Preferred embodiments will now be described more fully hereinafter with reference to the accompanying drawings. However, they may be embodied in different forms and should not be construed as limited to the embodiments set forth herein. Rather, these embodiments are provided so that this disclosure will be thorough and complete, and will fully convey the scope of the disclosure to those skilled in the art.

To solve the above-described problems, the present invention provides a pharmaceutical composition for preventing and treating periodontal diseases, comprising an extract of an Elm japonica plant as an active ingredient, and a health functional food for alleviating periodontal diseases, the preparation method of which is as follows.

### Example 1

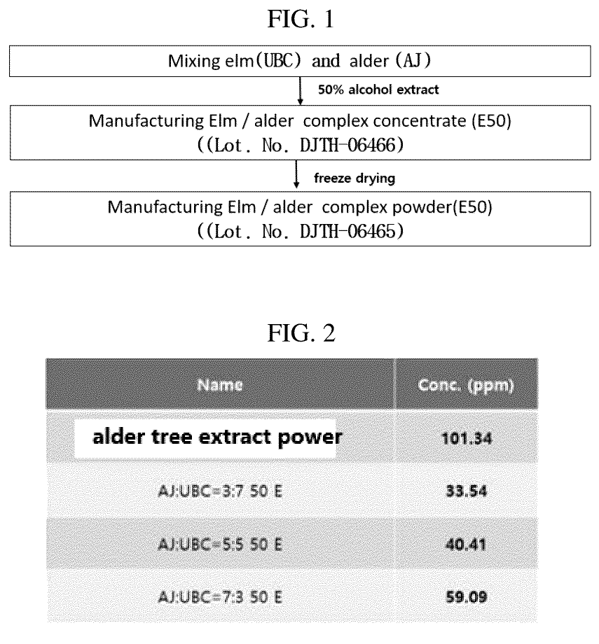

FIG. 1 is a step diagram of a method for obtaining a composite extract of Elm japonica (UBC) and Alnus japonica (AJ) according to an embodiment of the present invention. FIG. 1 illustrates a case in which the weight ratio is 1:1.

Referring to FIG. 1, in an exemplary embodiment of the present invention, for composite extraction of Ulmus macrocarpa and Alnus japonica, Elm japonica (UBC) and Alnus japonica (AJ) were mixed at a ratio of 3:7, 5:5, and 7:3, respectively. In FIG. 1, the weight ratio is 5:5, and the weight ratio of the Ulmus macrocarpa and the Alnus japonica tree is different in the raw material 300 kg.

50% alcohol-based 3,000 kg (1:10, w/w) was mixed with the prepared raw material 300 kg to perform extraction at 75±5° C. for 6 hours. After extraction, the mixture was cooled to room temperature, filtered through a 0.2 μm filter, and concentrated under reduced pressure to 60 Brix (55±5° C., 60 bar.), thereby obtaining a complex concentrate of Ulmus macrocarpa and Alnus japonica (E50) (Lot. No. DJTH-06466). After the concentration was completed, dextrin and purified water were mixed based on the concentrated solution and freeze-dried. After frozen and dried, 30 kg of extract powder of Ulmus macrocarpa and Alnus japonica (E50) was recovered (Lot No. DJTH-06465).

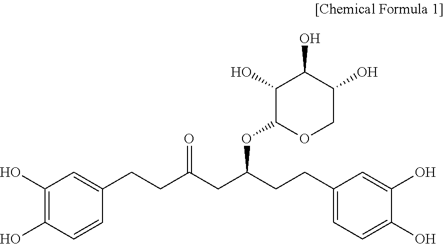

In addition, the Elm japonica extract includes catechin glycoside, 7-O-β-D-apiofuranoside (catechin 7-O-β-D-apiofuranoside) represented by the following Chemical Formula 2.

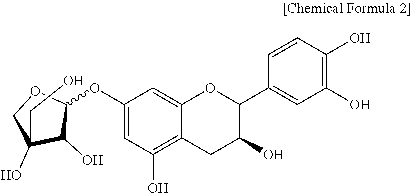

To this end, HPLC quantitative analysis of the extract powder and the extract powder of the Alnus japonica tree Elm japonica according to the ratio of the Alnus japonica tree to the extract was performed to confirm the content of oregonin, which is an indicator material of the Alnus japonica tree, for each sample.

FIG. 2 is a result of analyzing the content of oregonin of Chemical Formula 1 for each extract.

Referring to FIG. 2, all of the extracts according to an embodiment of the present invention are contained in oregonin.

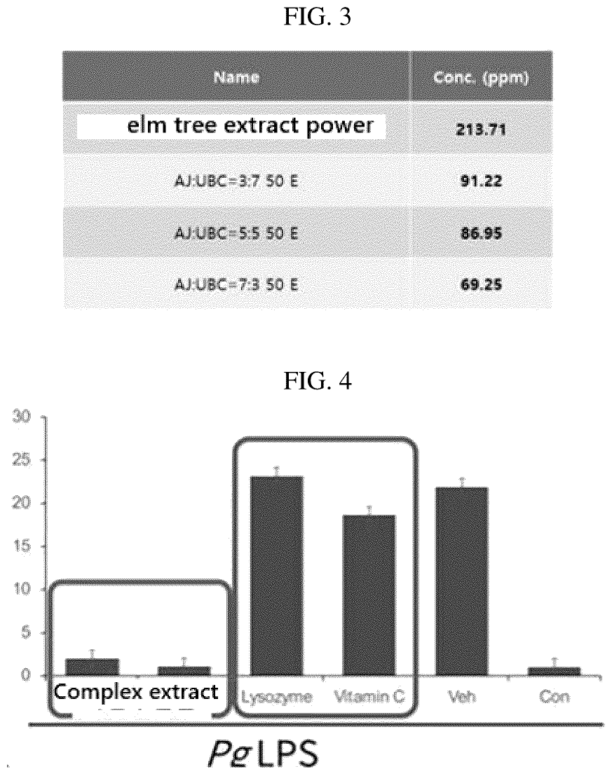

FIG. 3 is a result of analyzing the content of catechin glycosides of Chemical Formula 2 in each extract.

Referring to FIG. 3, all of the extracts according to an exemplary embodiment of the present invention were contained in the catechin glycoside of Chemical Formula 2.

### Experimental Example 1

### Periodontal Disease Improving Effects

In this experimental example, in order to confirm the treatment and improvement effects of periodontal disease, an inflammatory environment creation model for periodontal disease was first constructed according to the following method.

### 1) Analysis of an Inflammatory Cytokine Expression Level in a Cell

Endogenous inflammatory environment creation model

(1) Gingival fibroblasts are dispensed into 12 well plate with 1×105/well of density and cultured for 12 hours.

(2) Additional culture after treatment with 10 ng/ml of TNF-α and a test substance at a predetermined concentration.

(3) RNA extraction from cells cultured using total RNA extraction kit.

(4) Synthesis of cDNA Using Reverse transcription kit.

(5) Quantitative RT-PCR is performed using SYBR reagent, and in this case, a cycle threshold (Ct) value is derived using specific primer for three kinds of pro-inflammatory cytokine (TNF-α, IL-6, IL-1B, etc.).

(6) Analysis of Relative Expression Level of pro-inflammatory cytokine Using ΔΔCt Method
Model for creating an environment for extrinsic inflammation

(1) Gingival fibroblasts are dispensed into 12 well plate with 1×105/well of density and cultured for 12 hours.

(2) Treating oral bacteria (such as Porphyromonas gingivalis)-derived LPS (1 μg/ml) or extracellular vesicles with a test substance at a predetermined concentration, followed by further culture.

(3) RNA extraction from cells cultured using total RNA extraction kit.

(4) Synthesis of cDNA Using Reverse transcription kit.

(5) Quantitative RT-PCR is performed using SYBR reagent, and in this case, a cycle threshold (Ct) value is derived using specific primer for three kinds of pro-inflammatory cytokine (TNF-α, IL-6, IL-1β, etc.).

(6) Analysis of Relative Expression Level of pro-inflammatory cytokine Using ΔΔCt Method

2) Evaluation of intracellular inflammatory signal molecular mechanisms
Model for creating an endogenous inflammatory environment

(1) Gingival fibroblasts are dispensed into 12 well plate with 1×105/well of density and cultured for 12 hours.

(2) Additional culture after treatment with 10 ng/ml of TNF-α and a test substance at a predetermined concentration.

(3) Total protein extraction from the cultured cells by disruption of the cells with laemmli sample buffer followed by heating at sonication, and 95° C. for 5 minutes.

(4) After SDS-PAGE, transfer the protein in PVDF membrane and immerse membrane in 5% skim milk for 30 minutes to perform blocking.

(s) Quantification by binding antibodies specific to phospho NFκB, phospho STAT3, total NFκB and total STAT3, and then detecting chemiluminescence signals
Model for creating an extrinsic inflammatory environment

(1) Gingival fibroblasts are dispensed into 12 well plate with 1×105/well of density and cultured for 12 hours.

(2) Treating oral bacteria (such as Porphyromonas gingivalis)-derived LPS (1 μg/ml) or extracellular vesicles with a test substance at a predetermined concentration, followed by further culture.

(3) Total protein extraction from the cultured cells by disruption of the cells with laemmli sample buffer followed by heating at sonication, and 95° C. for 5 minutes.

(4) After SDS-PAGE, transfer the protein in PVDF membrane and immerse membrane in 5% skim milk for 30 minutes to perform blocking.

(5) Quantification by binding antibodies specific to phospho NFκB, phospho STAT3, total NFκB and total STAT3, and then detecting chemiluminescence signals

In this experimental example, the three natural extracts UBC60E, UBRF3, and UM/A 50E (see FIGS. 1 and 2) were used at a concentration of 1 to 100, and the effect of alleviating inflammation of fibroblasts was analyzed as follows.

### Inflammation Suppression Effect

### Analysis of the Expression Level of Inflammatory Cytokines in Cells (a Model for the Composition of an Endogenous Inflammatory Environment)

To this end, the natural extract was pretreated 1 hour before treatment with TNF-α or LPS, and RNA was extracted 4 hours after treatment with TNF-α or LPS to measure the expression level. At this time, as controls, lysozyme and batamine C, which are the main pharmaceutical ingredients of periodontal disease treatment (Igatan), were used.

FIG. 4 is a result of analyzing the effect of inhibiting the expression level of TNF-α of the extract according to the present invention by using lysozyme and batamine C, which are major pharmaceutical components of the existing commercialized therapeutic agent for periodontal diseases (Irgata), as controls.

Referring to FIG. 4, it can be seen that the complex extract according to an embodiment of the present invention has a significantly higher inhibitory effect on TNF-α expression than lysozyme and batamine C, which are the main components of the existing periodontal disease therapeutic agents.

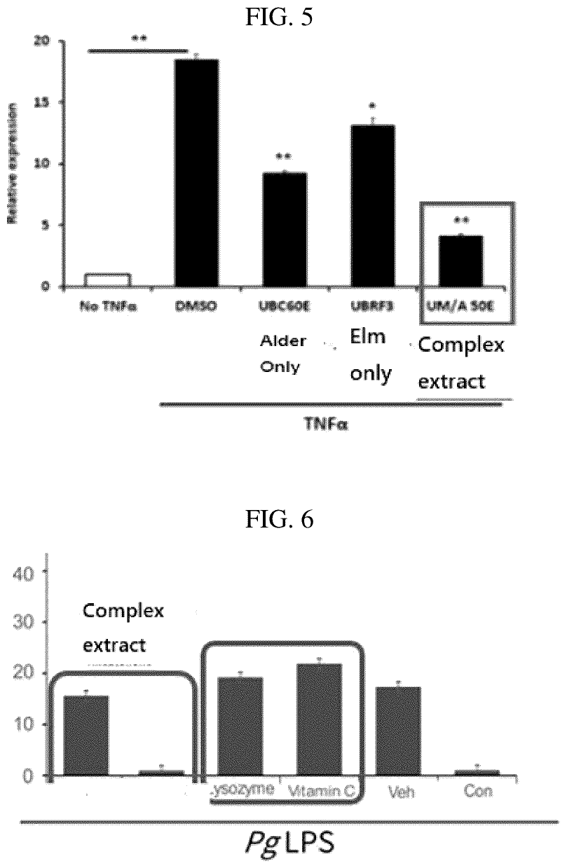

FIG. 5 is a result of an analysis of inhibition of TNF-α expression with a composite extract according to the present invention compared to a Alnus japonica alone (Alder only) and an Elm japonica alone (Elm only).

Referring to FIG. 5, it can be seen that the complex extract (Alnus japonica:Elm tree weight ratio=7:3) has a significantly higher effect of inhibiting the expression of TNF-α compared to the Alnus japonica alone and the Elm japonica alone.

This is a result of confirming a more excellent effect of increasing the ability to inhibit the expression of TNF-α when Catechin 7-O-β-D apiofuranoside, which is an Elm japonica-derived glycoside compound, and oregonin, which is a Japanese Alnus japonica-derived glycoside compound, are contained at the same time.

FIG. 6 shows the results of analysis of the IL-6 expression level of the extract according to the present invention using lysozyme and batamine C, which are the main pharmaceutical ingredients of the existing commercialized therapeutic agent for periodontal disease (Irgata), as controls.

Referring to FIG. 6, the complex extract according to an exemplary embodiment of the present invention has a higher inhibitory effect on IL-6 expression than lysozyme and batamine C, which are the main components of the existing periodontal disease therapeutic agents. In particular, it can be seen that the graph on the right side in the box (Alnus japonica:Elm japonica=7:3) has a remarkably high inhibitory activity against IL-6 expression.

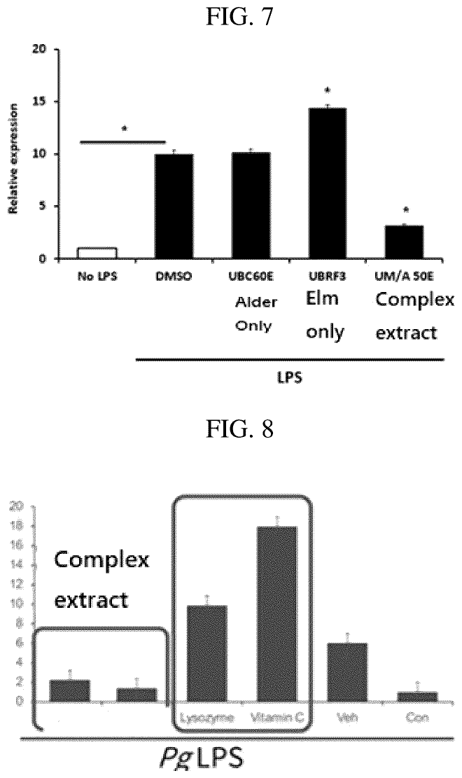

FIG. 7 shows the results of analyzing the expression level of IL-6 having a composite extract according to the present invention as compared with Alnus japonica alone and an Elm japonica alone.

Referring to FIG. 7, it can be seen that the complex extract (Alnus japonica: Elm tree weight ratio=7:3) has a significantly higher effect of inhibiting the expression of IL-6 compared to the Alnus japonica alone and the Elm japonica alone.

FIG. 8 shows the results of analysis of the expression level of IL-1β of the extract according to the present invention using lysozyme and batamine C, which are the main pharmaceutical ingredients of the existing commercialized therapeutic agent for periodontal disease (Irgata), as controls.

Referring to FIG. 8, the complex extract according to an exemplary embodiment of the present invention has a significantly higher inhibitory effect on the expression of IL-1β than lysozyme and batamine C, which are the main components of the existing periodontal disease therapeutic agents.

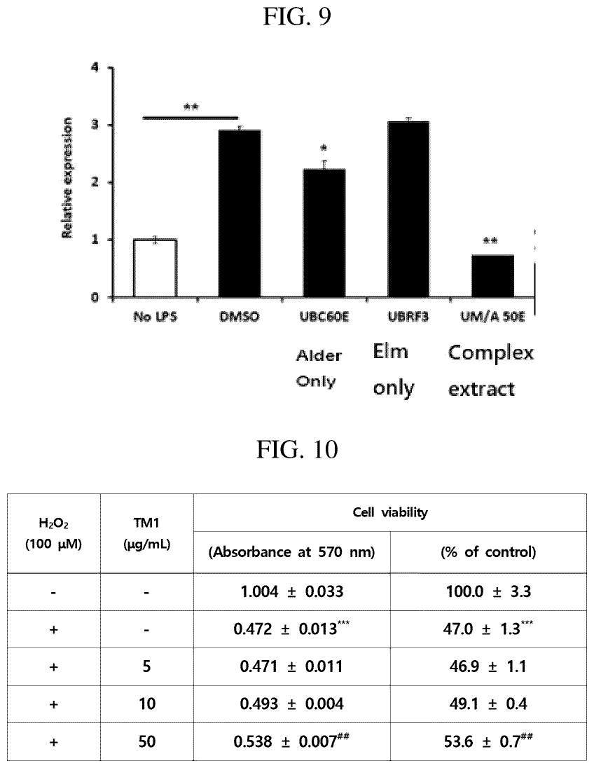

FIG. 9 shows the results of analysis of the expression level of IL-1β having a composite extract according to the present invention as compared with a Alnus japonica alone and an Elm japonica alone.

Referring to FIG. 9, it can be confirmed that the expression of IL-1β was statistically significantly suppressed in the composite sample containing both Catechin 7-O-β-D apiofuranoside, which is the glycoside compound derived from Elm japonica, and oregonin, which is the glycoside compound derived from Alnus japonica tree.

The present invention also provides a therapeutic agent for muscle loss and a health functional food comprising an extract of complex of alnus-elm japonica. Hereinafter, this will be described in more detail.

### Experimental Example 2

### Material

The elm japonica extract (U, TM1), Alnus japonica extract (A, TM2), elm-alnus complex extract-02 (elm: alder weight ratio 7:3, TM3), elm-alnus complex-03 (elm: alder weight ratio 5:5, TM4), and elm-alnus complex extract-04 (elm: alder weight ratio 3:7, TM5) were used as test materials, and the method of preparing the complex extract was the same as described in the experimental example 1

### Method

### Cell Culture

C2C12 cells, a myoblast derived from mouse skeletal muscle, were purchased from American Type Culture Collection (ATCC) and used. C2C12 cells were cultured in a 37° C. wet CO2 incubator (5% CO2/95% air) using a cell culture solution in which 10% fetal bovine serum (FBS), 100 units/mL penicillin, and 100 μg/mL streptomycin were added to Dulbecco's Modified Eagle Medium (DMEM). When the cells were 80% full of the culture dish, the cell monolayer was washed with phosphate buffer saline (PBS, pH 7.4), trypsin-2.65 mM EDTA was added thereto, and the cells were detached and subcultured, and the medium was exchanged every 2 days. To induce differentiation of C2C12 cells into myotubes, the cells were cultured by exchange with a myocyte differentiation culture medium in which 2% horse serum (HS) was added to DMEM medium, and the myocyte differentiation culture medium was exchanged every 2 days. The present invention is 2.2. the measurement of cell viability under normal conditions.

### Evaluation of Apoptosis of H2O2 Induced Myoblast

### C2C12 for Evaluating the Effect of a Test Substance on H2O2 Induced Myocyte Apoptosis

The cells were dispensed into 24-well plate at 5×104 cells/well and cultured for 24 hours. After culturing C2C12 cells for 24 hours, the cells were treated with 100 μM H2O2 to induce muscle cell damage, and the cells were cultured for 48 hours by treating five test substances with 100 μM H2O2 at various concentrations to investigate the effect of the test substance on protecting muscle cell damage. The degree of apoptosis of the myocytes was measured according to the method suggested by the manufacturer by using Cellular DNA Fragmentation ELISA kit (Sigma-Aldrich), which detects 5′-Bromo-2′-deoxy-uridine (BrdU)-labeled DNA.

### Measurement of a Protective Effect Against Dexamethasone Induced Myotube Cell Damage

C2C12 cells were seeded in 24-well plate at 5×104 cells/well and cultured for 24 hours. Thereafter, in order to induce differentiation into myotubes of C2C12 cells, the cell culture medium was exchanged with a myocyte differentiation culture solution to induce differentiation, thereby inducing differentiation for 4 days. Thereafter, 5 μM dexamethasone was treated to induce muscle cell atrophy, and in order to investigate the effect of protecting the muscle cell damage of the test substance, 5 test substances were treated with 5 μM dexamethasone at various concentrations to culture the cells for 24 hours. After culturing the cells for 24 hours, MTT assay was performed in the same manner as described above to measure a cell viability.

### Measurement of a Protective Effect Against Dexamethasone Induced Myotube Cell Atrophy

C2C12 cells were cultured in 24-well plate containing cover glass for 24 hours to a concentration of 5×104 cells/well. In order to induce differentiation into myotubes of C2C12 cells, the cell culture medium was exchanged with a myocyte differentiation culture medium to induce differentiation for 4 days. Thereafter, 5 UM dexamethasone was treated to induce muscle cell atrophy, and in order to investigate the effect of protecting the muscle cell damage of the test substance, five test substances were treated with 5 μM dexamethasone at various concentrations to culture the cells for 24 hours. The medium was removed, washed with PBS, and treated with 4% paraformaldehyde and 0.1% Triton X-100 to fix the cells. After blocking with 5% BSA/TBST, primary antibody step (MYH7, santa cruz) was performed. Thereafter, the tissue was stained with secondary antibody (Anti-mouse IgG-Alexa-594, ThermoFisher Scientifice), and then the expression of the protein was examined using an optical microscope (Carl Zeiss) by performing a control staining with 4′-6-Diamidino-2-phenylindole (DAPI, Sigma-Aldrich).

### Statistical Processing

All assay values were expressed as meantSEM. The collected results were analyzed using GraphPad Prism 5.0 (GraphPad software, San Diego, Calif., USA) program. Student's t-test and one-way analysis variance (ANOVA) were used to compare the difference between the test substance-treated group and the control group. It was judged to be statistically significant only when p<0.05 or higher.

### Result

### Effect on H2O2-Induced Myoblast Damage

H₂O₂ (hydrogen peroxide) is a strong oxidant that induces oxidative stress in the in vitro system. To investigate the effect of the five test substances on the muscle cell damage caused by H₂O₂ induction, the cell culture fluid of C2C12 cells was treated with 100 μM H₂O₂ to induce oxidative stress, and the five test substances were treated at various concentrations and cultured for 48 hours, and then the cell viability of C2C12 cells was measured.

FIGS. 10 to 14 show results of analyzing cell viability of TM1 to TM5, respectively.

Referring to FIGS. 10 to 14, the cell viability of the H2O2 treated group [H₂O₂ (+)/(−)] was significantly reduced compared to the control group [H₂O₂ (−)/(−)] not treated with H₂O₂.

As shown in FIG. 10, the treatment of TM1 (5, 10, 50 μg/mL) significantly increased the cell viability at the treatment concentration of 50 μg/mL compared to the H2O2 treatment group [H2O2 (+)/(−).

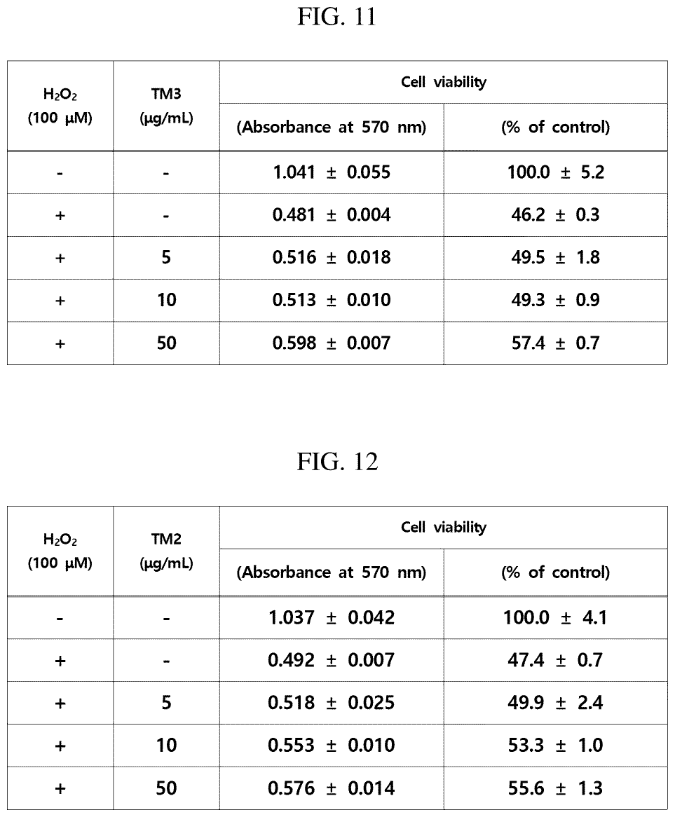

As shown in FIGS. 11 and 12, the treatment of TM2 and TM3 (5, 10, 50 μg/mL) significantly increased the cell viability from the treatment concentration of 10 μg/mL compared to the H2O2 treatment group [H₂O₂ (+)/(−).

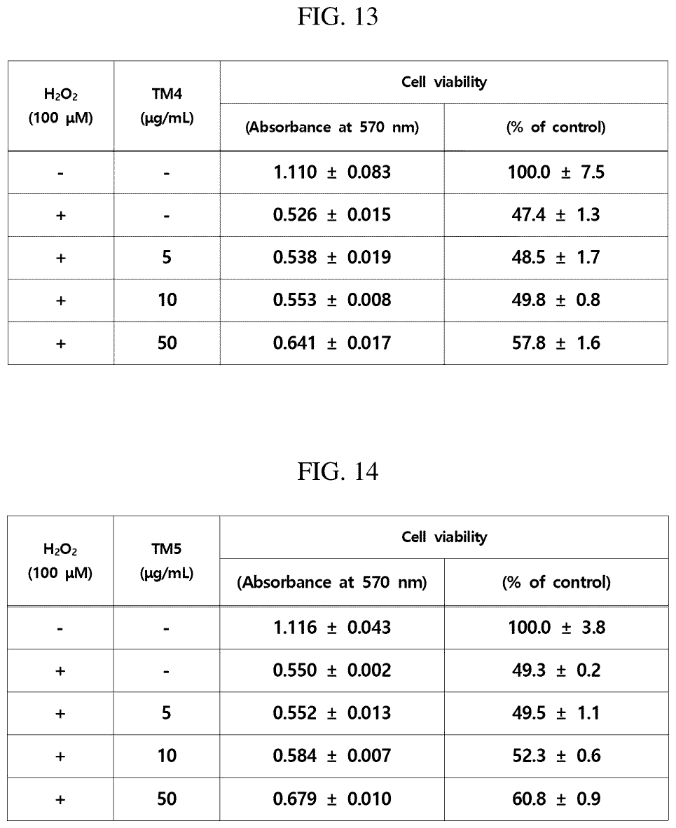

Also, according to FIG. 13, the treatment of TM4 (5, 10, 50 μg/mL) significantly increased the cell viability at the treatment concentration of 50 μg/mL compared to the H2O2 treatment group [H₂O₂ (+)/(−).

In FIG. 14, the treatment of TM5 (5, 10, 50 μg/mL) significantly increased the cell viability from the treatment concentration of 10 μg/mL compared to the H2O2 treatment group [H2O2 (+)/(−).

In addition, when each test substance was treated at a concentration of 50 μg/mL, cell viability increased by 14.0% (TM1), 17.3% (TM2), 24.2% (TM3), 21.9% (TM4), and 23.3% (TM5), respectively, compared to the H₂O₂ treatment group [H2O2 (+)/(−).

Effect on H₂O₂ Induced Myoblast Cell Apoptosis

Oxidative stress is well known to cause cell death by causing DNA damage, and cell death by such oxidative stress as H2O2 occurs through a apoptosis process, which is a kind of programmed cell death. Therefore, in the present invention, in order to evaluate the effect of five test substances on apoptosis by oxidative stress, segmented DNA was quantified using Cellular DNA Fragmentation ELISA kit.

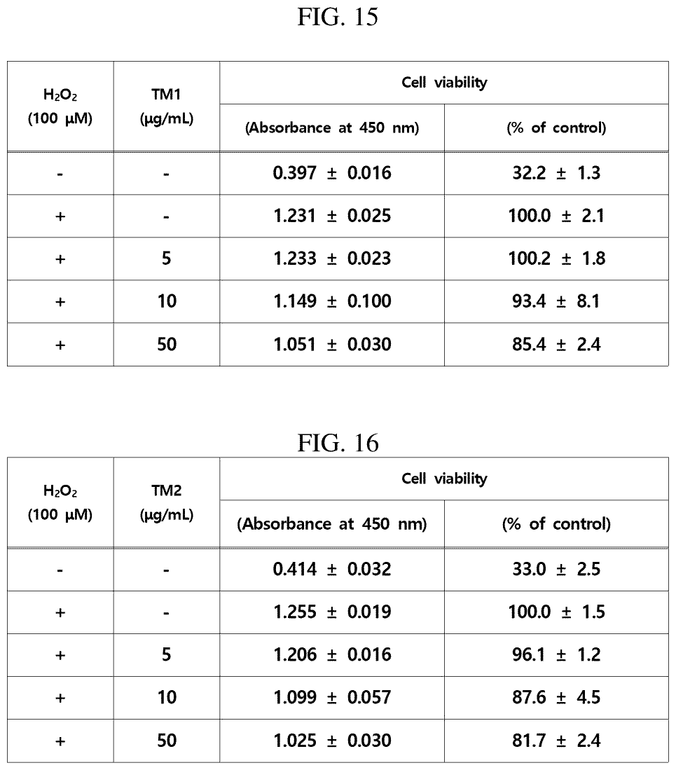

FIGS. 15 to 19 are results of analyzing effects of TM1 to TM5 on apoptosis, respectively.

Referring to FIGS. 15 to 19, compared with the control group [H₂O₂ (−)/(−)] not treated with H₂O₂, the H₂O₂ treatment group [H₂O₂ (+)/(−)] significantly increased apoptosis. In comparison with the H₂O₂ treatment group [H₂O₂ (+)/(−)], the treatment with TM1, the apoptosis was reduced by 14.6% at the highest treatment concentration of 50 μg/mL (FIG. 15).

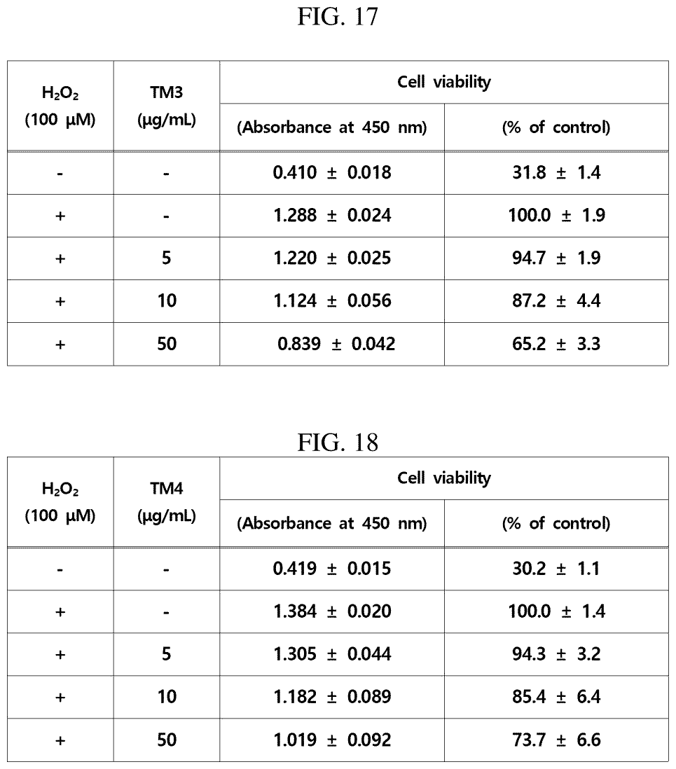

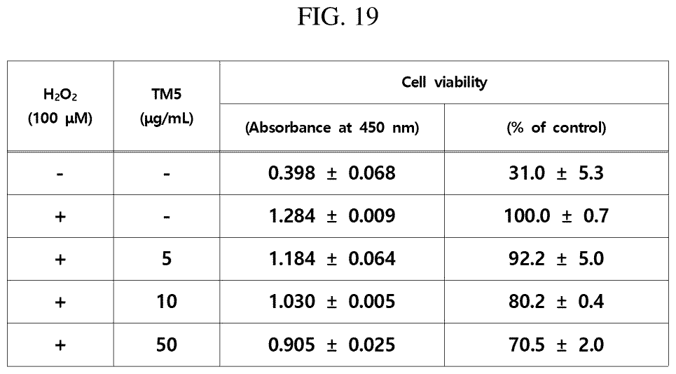

Also, the treatment with TM2, the apoptosis was significantly reduced from the highest treatment concentration of 50 μg/mL (FIG. 16). The treatment with TM3, the apoptosis was significantly reduced from the highest treatment concentration of 50 μg/mL (FIG. 17). In case of TM4 treatment, the apoptosis was reduced by 26.3% at the highest treatment concentration of 50 μg/mL (FIG. 18). TM5 also reduces the apoptosis significantly. (FIG. 19).

### Effect on Dexamethasone Induced Myotube Cell Damage

Dexamethasone is one of the representative glucocorticoid, which causes the decomposition of skeletal muscle in clinical misuse, and based on this, it is widely used to induce muscle cell atrophy in the in vitro system. To investigate the effect of the five test substances on the muscle cell damage caused by glucocorticoid, C2C12 cells were cultured with a muscle cell differentiation culture solution for 4 days to induce differentiation into myotubes, and then 5 μM dexamethasone was treated to induce muscle cell atrophy, and the viability of the myotubes was measured by treating the five test substances.

Treatment with 5 μM dexamethasone induced myocyte atrophy, and it was observed that the diameter of myocytes was significantly reduced compared to the control group [DEX (−)/(−)] not treated with dexamethasone.

However, diameter was significantly increased compared to the DEX treatment group [DEX (+)/(−)] after treatment with 5 types of test substances.

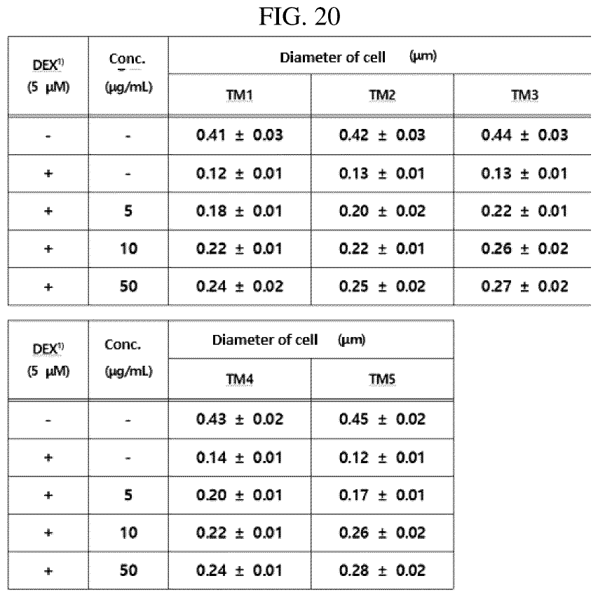

FIG. 20 shows the results of the diameter of muscle cells for dexamethasone-treated muscular cells.

Referring to FIG. 20, when measuring the length of the diameter at different concentrations (conc.), the diameter of the DEX-treated group [DEX (+)/(−)] was significantly reduced to 0.12 to 0.14 μm, compared to the diameter of the control group [DEX (−)/(−)] not treated with dexamethasone. The diameter of the muscle cell, however, significantly increased for all five types of test substances. The diameter of the muscle cells at the highest treatment concentration of 50 μg/mL of the five test substances increased by 2.00 times (TM1), 1.92 times (TM2), 2.07 times (TM3), 1.71 times (TM4), and 2.33 times (TM5), respectively, compared to the DEX-treated group [DEX (+)/(−).

In summary, the complex extract comprising elm and Alnus japonica can significantly increase the cell viability of C2C12 cells (myoblasts), which were significantly reduced by H2O2 treatment. In addition, TM5, which is a complex extract, increased the muscle cell diameter reduced by dexamethasone treatment to a level of 2.33 times shows an effect of protecting excellent muscle cell atrophy of the complex extract according to the present invention compared to each unit extract. Therefore, it is possible to confirm the possibility of future muscle reduction (atrophy) treatment and development of improved functional pharmaceutical materials.

As used herein, the term “comprising as an active ingredient” means that the extract of the present invention comprises an amount sufficient to achieve the prevention and treatment efficacy of muscle loss.

In the present specification, the term “periodontal disease” means any disease occurring in periodontal tissue, and the term “prevention” used in the present invention means all actions of inhibiting periodontal disease or delaying the occurrence of periodontal disease by administration of the pharmaceutical composition according to the present invention. In addition, the term “treatment” used in the present invention refers to all actions in which the symptoms of periodontal disease are improved or are beneficially changed by administration of the pharmaceutical composition according to the present invention. As used herein, the term “amelioration” refers to any action that at least reduces a parameter related to a condition being treated, for example, the degree of symptoms. In this case, the functional food composition can be used simultaneously with or separately from medicines for treatment before or after the onset of the disease to prevent or ameliorate the periodontal disease

The pharmaceutical composition for preventing and treating periodontal diseases according to the present invention may further include a pharmaceutically acceptable carrier, excipient, or diluent.

The pharmaceutically acceptable carrier in the composition of the present invention is commonly used in formulation, and includes lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methylcellulose, methyl hydroxybenzoate, propyl hydroxybenzoate, talc, magnesium stearate, mineral oil, and the like, but is not limited thereto. The pharmaceutical composition of the present invention may further include a lubricant, a wetting agent, a sweetening agent, a flavoring agent, an emulsifying agent, a suspending agent, a preservative, and the like, in addition to the above ingredients.

The pharmaceutical composition of the present invention may be administered orally or parenterally, and in the case of parenteral administration, it may be administered by intravenous injection, subcutaneous injection, intramuscular injection, intraperitoneal injection, transdermal administration, etc.

A suitable dosage of the pharmaceutical composition of the present invention varies depending on factors such as formulation method, administration method, patient age, body weight, sex, pathological condition, food, administration time, administration route, excretion rate, and response sensitivity, and usually a skilled doctor can easily determine and prescribe a dosage effective for the desired treatment or prevention.

The pharmaceutical composition for preventing and treating periodontal diseases of the present invention can be used as an active ingredient by containing other pharmaceutically active ingredients in addition to extracts of Elm japonica plants, or by being mixed with a pharmaceutical composition containing other active ingredients.

The food or health functional food composition of the present invention may further include a sitologically acceptable food supplement additive. Sitologically acceptable food additives that can be used in the present invention include, but are not limited to, sugars such as glucose, fructose, maltose, sucrose, dextrin, and cyclodextrin; natural carbohydrates such as xylitol, sorbitol, and erythritol; natural flavoring agents such as thaumatin and stevia extract; synthetic flavoring agents such as saccharin and aspartame; coloring agents; pectic acid or a salt thereof; alginic acid or a salt thereof; organic acids; protective colloidal thickeners; pH adjusters; stabilizers; preservatives; glycerin; alcohols; and carbonating agents. The food composition of the present invention may be in a form selected from the group consisting of powders, granules, tablets, capsules, candies, chewing gum, jelly, and beverages. The content of the plant extract of the genus Elm japonica in the food composition may be appropriately selected in consideration of the shape, flavor, taste, and the like of the food, and may be, for example, in the range of 0.01 to 30% by weight based on the total weight of the food. It will be apparent to those skilled in the art that the form, composition, preparation method, and the like of the food composition according to the present invention may be appropriately selected from those skilled in the art.

Therefore, the composition comprising the extract of Elm japonica plants as an active ingredient according to the present invention can be used as a toothpaste for humans and animals, as well as a pharmaceutical composition or a food composition, and as a feed for animals such as companion animals.

In addition, the extract according to the present invention can be used as an active ingredient for toothpaste and mouthwash.

While the present disclosure has been described with reference to the embodiments illustrated in the figures, the embodiments are merely examples, and it will be understood by those skilled in the art that various changes in form and other embodiments equivalent thereto can be performed. Therefore, the technical scope of the disclosure is defined by the technical idea of the appended claims.

The drawings and the forgoing description gave examples of the present invention. The scope of the present invention, however, is by no means limited by these specific examples. Numerous variations, whether explicitly given in the specification or not, such as differences in structure, dimension, and use of material, are possible. The scope of the invention is at least as broad as given by the following claims.

What is claimed is:

1. A pharmaceutical composition for preventing and treating sarcopenia and periodontal disease, comprising extracts of Alnus japonica and Elm japonica as active ingredients.

2. The pharmaceutical composition for preventing and treating sarcopenia and periodontal disease of claim 1, wherein the Elm japonica extract comprises catechin 7-O-β-D-apiofuranoside (catechin 7-O-β-D-apiofuranoside).

3. The pharmaceutical composition for preventing and treating sarcopenia and periodontal disease of claim 1, wherein the Alnus japonica extract comprises oregonin.

4. The pharmaceutical composition for preventing and treating sarcopenia and periodontal disease of claim 1, wherein a weight ratio of the extract of Alnus japonica and the extract of Elm japonica is 3:7 to 7:3.

5. A health functional food for preventing and improving periodontal disease, comprising an extract of Alnus japonica and Elm japonica at the same time as an active ingredient.

6. The health functional food for preventing and improving periodontal disease of claim 5, wherein the Elm japonica extract comprises catechin 7-O-β-D-apiofuranoside (catechin 7-O-β-D-apiofuranoside).

7. The health functional food for preventing and improving periodontal disease of claim 5, wherein the Alnus japonica extract comprises oregonin.

8. The health functional food for preventing and improving periodontal disease of claim 5, wherein a weight ratio of the extracts of Alnus japonica and Elm japonica is 3:7 to 7:3.

9. A feed comprising extracts of Alnus japonica and Elm japonica as active ingredients at the same time.

10. A toothpaste comprising extracts of Alnus japonica and Elm japonica as active ingredients at the same time.

11. A mouthwash comprising an extract of Alnus japonica and Elm japonica as an active ingredient at the same time.
